(19) United States
(12) Patent Application Publication
(10) Pub. No.: US 2026/0275405 A1
(43) Pub. Date: Sep. 17, 2026

(54) MASS TAGGING NUCLEIC ACIDS FOR QUALITY ASSURANCE AND CONTROL

(71) Applicant: The University of South Alabama, Mobile, AL (US)

(72) Inventors: Marie Migaud, Mobile, AL (US); Faisal Hayat, Mobile, AL (US)

(73) Assignee: The University of South Alabama, Mobile, AL (US)

(21) Appl. No.: 19/568,272

(22) Filed: Mar. 16, 2026

Related U.S. Application Data
(60) Provisional application No. 63/772,179, filed on Mar. 14, 2025.

(51) Int. Cl. C12Q 1/6806; C07K 14/005; C12Q 1/6872; G01N 30/72; G01N 33/58; H01J 49/16; H01J 49/40

(52) U.S. Cl. CPC C12Q 1/6806; C07K 14/005; C12Q 1/6872; G01N 30/7233; G01N 33/58; H01J 49/164; H01J 49/40; G01N 2458/15

(57) ABSTRACT

The present disclosure provides a system and method for conducting quality control of nucleic acid synthesis by mass-tagging nucleic acid building blocks. The method includes generating isotopically labeled nucleosides using mechanochemical synthesis, converting the isotopically labeled nucleosides to nucleotides or nucleoside phosphoramidites, and incorporating the nucleotides or nucleoside phosphoramidites into oligonucleotides. The method further includes analyzing the oligonucleotides using mass spectrometry to determine nucleic acid composition and detect contaminants. The mass-tagged nucleic acid building blocks enable improved quality control and authentication of RNA products.

### CROSS REFERENCES TO RELATED APPLICATIONS

This application claims the benefit of U.S. Provisional Application No. 63/772,179, filed on Mark 14, 2025, which is herein incorporated entirely by reference.

### FIELD OF INVENTION

The present disclosure relates to quality control of synthetic nucleic acids, and more particularly to mass-tagged nucleotides and oligonucleotides for authentication and integrity validation of RNA products by mass spectrometry.

### BACKGROUND

Oligonucleotides have emerged as powerful tools for gene editing, therapeutic agent engineering, and uncovering life processes. These synthetic nucleic acid molecules can be designed to target specific genetic sequences, modulate gene expression, or serve as building blocks for complex biological systems. The applications of oligonucleotides span a wide range of fields, including medicine, agriculture, and biotechnology. As the use of oligonucleotides in research and therapeutic applications continues to grow, there is an increasing demand for high-quality synthetic nucleic acid products. The purity and integrity of manufactured oligonucleotides are paramount to their effectiveness and safety in various applications, particularly for mRNA-based therapeutics and vaccines. However, ensuring consistent quality across batches of synthetic nucleic acids remains a significant challenge in the field, as synthetic RNA molecules are notoriously unstable and sensitive to degradation by environmental factors such as temperature fluctuations, RNase contamination, and oxidative stress.

Current quality control methods for oligonucleotides often rely on techniques such as high-performance liquid chromatography (HPLC), capillary electrophoresis, and UV spectroscopy. While these methods provide valuable information about the overall purity and yield of oligonucleotide products, they have notable limitations in detecting low-abundance contaminants or subtle structural modifications that can impact functionality. For instance, HPLC may fail to distinguish between full-length products and truncated sequences that differ by only a few nucleotides. Capillary electrophoresis, while useful for size-based separation, cannot readily identify chemical modifications to nucleobases or sugar moieties. UV spectroscopy provides only aggregate measurements of nucleic acid concentration without revealing sequence-specific information or the presence of degradation products. These analytical gaps become particularly problematic when assessing the quality of therapeutic-grade mRNA products, where even minor impurities or structural defects can compromise efficacy or safety.

Mass spectrometry has emerged as a powerful analytical tool for characterizing biomolecules, including nucleic acids, offering high sensitivity and the ability to provide detailed structural information. Techniques such as liquid chromatography-mass spectrometry (LC-MS) and matrix-assisted laser desorption/ionization time-of-flight mass spectrometry (MALDI-TOF MS) can detect molecular masses with high precision, enabling identification of specific nucleotide compositions. However, the application of mass spectrometry for routine quality control of oligonucleotide products has been limited by factors such as the complexity of data interpretation and the lack of standardized protocols. Distinguishing synthetic oligonucleotides from naturally occurring nucleic acid contaminants presents a particular challenge, as both share identical chemical compositions. Furthermore, current mass spectrometry approaches lack reliable internal standards that would enable quantitative assessment of product integrity across different structural regions of complex RNA molecules, such as the 5′ cap, coding sequence, and poly-A tail. The absence of such standards makes it difficult to authenticate synthetic products or detect contamination from biological sources.

The incorporation of stable isotopes into nucleic acid building blocks has been explored as a strategy for enhancing the detection and quantification of specific molecules in complex biological samples. Isotopic labeling can provide unique mass signatures that facilitate the identification and tracking of target compounds, as isotopes such as ¹⁸O, ¹³C, and IN produce detectable mass shifts without altering chemical behavior. However, the widespread use of isotopically labeled nucleic acids has been hindered by the high costs and technical challenges associated with their synthesis using conventional solution-phase chemistry. Traditional synthetic routes for isotopically labeled nucleosides require extensive protecting group manipulations, multiple purification steps, and expensive isotope-enriched reagents. These factors have limited the practical application of isotope-labeled nucleic acids for quality control purposes at industrial scales. As the field of nucleic acid-based technologies continues to advance, there remains a growing need for cost-effective approaches to producing traceable nucleic acid building blocks that could enable improved quality assurance and authentication of synthetic oligonucleotide products.

### SUMMARY

A system and method for conducting quality control of industrial nucleic acid synthesis is provided. In one aspect, the present invention is a composition comprising mRNA with mass-labeled elements. In another aspect, the present invention is an artificial isotopic ratio within a synthetic mRNA composition which allows it to be distinguished from naturally occurring mRNA or contamination. In yet another aspect, the present invention is a method of analyzing the molecular mass of an mRNA composition to quantify its ratio of mass-labeled components. In still another aspect, the present invention is a method of detecting contamination in an mRNA composition based on its ratio of mass-labeled mRNA components. Generally, the present invention permits quality control and quality assurance of nucleic acids at the industrial level by comparing mass-labeled polynucleotides to internal standards.

The system and method may comprise a composition that includes a synthetic RNA molecule comprising one or more isotopically labeled nucleosides incorporated at particular loci on the RNA molecule. The one or more isotopically labeled nucleosides can include an ¹⁸O-labeled nucleoside, though other isotopic labels may alternatively be employed. The synthetic RNA molecule may be an mRNA molecule, and as such the one or more isotopically labeled nucleosides can be incorporated in a 5′ cap structure, a coding sequence, or a 3′ poly-A tail of the synthetic RNA molecule. The composition may optionally further include a plurality of unlabeled nucleosides, wherein the ratio of isotopically labeled nucleosides to unlabeled nucleosides can be between 1:10 and 1:1000. Alternatively, other ratios may be selected based on the particular application or detection methodology employed. The isotopic labeling approach can provide a means for distinguishing synthetic nucleic acids from naturally occurring counterparts during quality assessment procedures.

The method may include one or more of the following features, which can be combined or modified according to particular implementations. The mechanochemical conditions under which the mass-labeled nucleotides are synthesized may comprise ball milling, though other mechanochemical approaches can alternatively be utilized. The method may optionally further include purifying the isotopically labeled nucleoside triphosphate by C18-reversed phase chromatography or other suitable purification techniques. The isotopically labeled nucleoside triphosphate can be an ¹⁸O-labeled nucleoside triphosphate, though other isotopic labels may alternatively be incorporated. The method may further include using the isotopically labeled nucleoside triphosphate as a substrate for RNA polymerase to incorporate the isotopically labeled nucleoside into an RNA molecule. The RNA molecule can be an mRNA molecule but may alternatively be another RNA molecule useful in industrial, medical, or research processes.

Quality control and quality assurance of the synthetic RNA molecule may be enacted by comparing the detected quantity of the one or more isotopically labeled nucleosides to an expected quantity to assess the makeup of the synthetic RNA molecule. This can be determined using mass spectrometry (MS) to detect changes in molecular mass from the incorporation of ¹⁸O or other isotopic labels. The method may optionally further include detecting the presence of contaminants or degradation products in the synthetic RNA molecule based on the MS analysis. The mass spectrometry analysis can comprise liquid chromatography-mass spectrometry (LC-MS) or alternatively matrix-assisted laser desorption/ionization time-of-flight mass spectrometry (MALDI-TOF MS). Other analytical techniques may optionally be employed in conjunction with or as alternatives to these mass spectrometry approaches. The disclosed methods and compositions can facilitate authentication and integrity validation of synthetic nucleic acid products across various applications.

The foregoing summary has outlined some features of the biologically functional polynucleotide and method of production so that those skilled in the pertinent art may better understand the detailed description that follows. Additional features that form the subject of the claims will be described hereinafter. Those skilled in the pertinent art should appreciate that they can readily utilize these features for designing or modifying other structures for carrying out the same purpose of the biologically functional polynucleotide and method disclosed herein. Those skilled in the pertinent art should also realize that such equivalent designs or modifications do not depart from the scope of the biologically functional polynucleotide and method of the present disclosure.

### BRIEF DESCRIPTION OF FIGURES

These and other features, aspects, and advantages of the present disclosure will become better understood with regard to the following description, appended claims, and accompanying drawings where:

FIG. 1 illustrates a synthetic mRNA molecule with labeled nucleosides consistent with the principles of the present disclosure.

FIG. 2 shows chemical synthesis schemes for nucleosides using mechanochemical methods, consistent with the principles of the present disclosure.

FIG. 3 shows a chemical reaction scheme for synthesizing isotopically labeled nucleosides and nucleotides, consistent with the principles of the present disclosure.

FIG. 4 depicts an analytical workflow for processing and analyzing tagged nucleic acids, consistent with the principles of the present disclosure.

FIG. 5 illustrates a synthesis and analysis workflow for mass-tagged RNA molecules, consistent with the principles of the present disclosure.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For instance, where a particular feature is disclosed in the context of a particular aspect or embodiment of the invention, or a particular claim, that feature can also be used, to the extent possible, in combination with/or in the context of other particular aspects of the embodiments of the invention, and in the invention generally.

### Exemplary Definitions

In accordance with long standing patent law convention, the words “a” and “an” when used in this application, including the claims, denote “one or more.”

The term “comprises” and grammatical equivalents thereof are used herein to mean that other components, steps, etc. are optionally present. For instance, a biologically functional polynucleotide “comprising” components A, B, and C can contain only components A, B, and C, or can contain not only components A, B, and C, but also one or more other components.

The term “consisting of” and grammatical equivalents thereof are used herein to mean that other components, steps, etc. are not optionally present. For instance, a biologically functional polynucleotide “consisting of” components A, B, and C can only contain components A, B, and C.

The terms “about” and “approximately,” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

The term “for instance” or “e.g.,” as used herein, is used merely by way of example, without limitation intended, and should not be construed as referring only those items explicitly enumerated in the specification.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

In accordance with the present invention, polynucleotides, nucleic acids, nucleic acid segments, and nucleic acid sequences include, without limitation, DNA, RNA, peptide nucleic acids (PNAs), and fragments, analogs, derivatives, and modified forms thereof, whether obtained from natural sources or prepared, synthesized, or modified in whole or in part by human intervention.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defined below:

As used herein, the term “nucleic acid” includes one or more types of: polydeoxyribonucleotides containing 2-deoxy-D-ribose (“DNA”), polyribonucleotides containing D-ribose (“RNA”), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, but are not limited to, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

As used herein, the term “nucleotide” refers to a molecule comprising a nucleobase, a sugar moiety, and one or more phosphate groups. The nucleobase may be a purine or pyrimidine, and the sugar moiety may be ribose or deoxyribose. Nucleotides include naturally occurring and synthetic or modified nucleotides. As used herein, the term “nucleoside” refers to a molecule comprising a nucleobase and a sugar moiety and lacking a phosphate group.

In addition, “nucleotide analogs” as used herein refer to modified or non-naturally occurring nucleotides, including but not limited to analogs having altered stacking interactions such as 7-deaza purines, base analogs having alternative hydrogen-bonding configurations, non-hydrogen-bonding analogs, “universal” bases such as 5-nitroindole and 3-nitropyrrole, and modified nucleotides having sugar modifications such as dideoxy nucleotides and 2′-O-methyl nucleotides. Nucleotide analogs include modified forms of both ribonucleotides and deoxyribonucleotides.

As used herein, the term “mRNA” or “messenger RNA” refers to a single-stranded ribonucleic acid molecule that carries genetic information from DNA to the ribosome, where it specifies the amino acid sequence of the protein products of gene expression. mRNA may include a 5′ cap structure, a 5′ untranslated region (UTR), a coding sequence, a 3′ UTR, and a 3′ poly(A) tail. The mRNA may be naturally occurring or synthetic and may include modifications to one or more of its structural components. Such modifications may include, but are not limited to, alterations of the nucleotide bases, sugar moieties, or internucleotide linkages, as well as the incorporation of non-canonical nucleotides or nucleotide analogs.

As used herein, the term “transcription” refers to the process by which genetic information encoded in a segment of DNA is copied into a complementary RNA molecule. This process is most commonly carried out by RNA polymerase enzymes and may involve the synthesis of various types of RNA, including mRNA, transfer RNA (tRNA), ribosomal RNA (rRNA), and non-coding RNAs. Transcription may occur in both prokaryotic and eukaryotic cells and may be subject to regulation by various factors such as transcription factors, enhancers, and repressors. The resulting RNA transcript may undergo further processing, including splicing, capping, and polyadenylation, before it is utilized by the cell for protein synthesis or other cellular functions.

As used herein, the term “translation” refers to the process by which the genetic code carried by mRNA is decoded to produce a specific polypeptide according to the rules specified by the genetic code. This process may occur in the cytoplasm of cells and may involve ribosomes, tRNAs, and various protein factors. Translation usually begins with the binding of the small ribosomal subunit to the mRNA, followed by the sequential addition of amino acids to the growing polypeptide chain as specified by the codons in the mRNA sequence. The process typically terminates when the ribosome encounters a stop codon. Translation may be subject to regulation at various stages and may be influenced by factors such as the availability of amino acids, the presence of regulatory proteins, and cellular energy levels. The resulting polypeptide often undergoes further modifications before becoming a functional protein.

As used herein, the term “open reading frame” or “ORF” refers to a continuous coding region of a nucleic acid molecule that begins with a start codon and ends with a stop codon and encodes a polypeptide. In the context of an mRNA molecule, the ORF is distinct from untranslated regions and the poly(A) tail.

As used herein, the term “5′ guanosine cap” refers to a modified guanosine nucleotide attached to the 5′ end of an RNA molecule through a 5′-5′ triphosphate linkage. The 5′ guanosine cap may include methylation at one or more positions, such as methylation at the N7 position of guanine or methylation at the 2′-O position of one or more adjacent nucleotides, including cap 0, cap 1, and cap 2 structures. Such cap structures may contribute to mRNA stability and translation.

As used herein, the term “poly(A) tail,” also referred to herein as a “poly-A tail,” refers to a sequence of adenosine nucleotides at the 3′ end of an RNA molecule, typically an mRNA molecule. The poly(A) tail may be naturally occurring or synthetically added and may contribute to RNA stability, translation efficiency, and other functional properties of the RNA molecule.

As used herein, the term “DNA source” refers to any DNA fragment carrying a suitable polymerase promoter, internal translation initiation site, transgene, untranslated regions or linkers, enhancers, repressors and any other regulatory elements needed, including nucleotide sequences for hairpins, to create suitable mRNA transcripts. Alternatively, a DNA molecule that has been isolated free of total genomic DNA of a particular species may serve as a “DNA source.” Therefore, a DNA source obtained from a biological sample using one of the compositions disclosed herein may refer to one or more DNA sources that may or may not have been isolated away from, or purified free from, total genomic DNA of the particular species from which they are obtained. Included within the term “DNA source,” are DNA segments and smaller fragments of such segments, as well as recombinant vectors, including, for instance, plasmids, cosmids, phage, viruses, PCR product, synthetic DNA, DNA ligation products and the like.

The terms “identical” or percent “identity,” in the context of two or more nucleic acid or polypeptide sequences, refer to sequences or subsequences that are the same or that share a specified percentage of nucleotides or amino acid residues when aligned for comparison using methods known in the art.

The term “naturally occurring” as used herein as applied to an object refers to the fact that an object can be found in nature. For instance, a polypeptide or polynucleotide sequence that is present in an organism (including viruses) that can be isolated from a source in nature and which has not been intentionally modified by the hand of man in a laboratory is naturally occurring. As used herein, laboratory strains of rodents that may have been selectively bred according to classical genetics are considered naturally occurring animals.

The term “operably linked,” as used herein, refers to the arrangement of nucleic acid sequences in a functional relationship such that one sequence affects the transcription, processing, translation, or expression of another sequence. In some embodiments, operably linked sequences are contiguous; in other embodiments, they are non-contiguous but still functionally associated.

The phrase “pharmaceutically acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, but are not limited to, acid addition salts formed with inorganic acids (e.g., hydrochloric acid, hydrobromic acid, sulfuric acid, phosphoric acid, nitric acid, and the like); and salts formed with organic acids including, but not limited to, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for instance, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For instance, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “1” isomeric form. However, residues in the “d” isomeric form may be substituted for any 1-amino acid residue provided the desired properties of the polypeptide are retained.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about 2 to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post-translationally modified and include any sugar or other derivative(s) or conjugate(s) added to the backbone amino acid chain.

The term “recombinant” indicates that the material (e.g., a polynucleotide or a polypeptide) has been artificially or synthetically (non-naturally) altered by human intervention. The alteration can be performed on the material within or removed from, its natural environment or state.

As used herein, the terms “complementary” or “complementarity” are used in reference to polynucleotides (i.e. a sequence of nucleotides such as an oligonucleotide or a target nucleic acid) related by the base-pairing rules. For instance, for the sequence “5′-A-G-T-3′,” is complementary to the sequence “3′-T-C-A-5′.” Complementarity may be “partial,” in which only some of the nucleic acids' bases are matched according to the base pairing rules. Or there may be “complete” or “total” complementarity between the nucleic acids. The degree of complementarity between nucleic acid strands has significant effects on the efficiency and strength of hybridization between nucleic acid strands. This is of particular importance in detection methods that depend upon binding between nucleic acids. Either term may also be used in reference to individual nucleotides, especially within the context of oligonucleotides. For instance, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

The term “substantially complementary,” when used to define nucleic acid sequences, means that a particular sequence is sufficiently complementary to all or a portion of a selected DNA or RNA sequence to hybridize specifically under appropriate conditions. The number of mismatches that may be tolerated depends on the sequence length, base composition, and hybridization conditions. In some embodiments, complete complementarity may be preferred.

The phrases “isolated” or “biologically pure” refer to material that is substantially, or essentially, free from components that normally accompany the material as it is found in its native state. Thus, isolated polynucleotides in accordance with the invention preferably do not contain materials normally associated with those polynucleotides in their natural, or in situ, environment.

“Link” or “join” refers to any method known in the art for functionally connecting one or more proteins, peptides, nucleic acids, or polynucleotides, including, but not limited to, recombinant fusion, covalent bonding, disulfide bonding, ionic bonding, hydrogen bonding, electrostatic bonding, and the like.

As used herein, the term “plasmid” refers to a genetic construct that is composed of genetic material (i.e., nucleic acids). Typically, a plasmid contains an origin of replication that is functional in bacterial host cells, e.g., Escherichia coli, and selectable markers for detecting bacterial host cells including the plasmid. Plasmids of the present invention may include one or more genetic elements as described herein arranged such that an inserted coding sequence can be transcribed and translated in a suitable expression cell. In addition, the plasmid may include one or more nucleic acid segments, genes, promoters, enhancers, activators, multiple cloning regions, or any combination thereof, including segments that are obtained from or derived from one or more natural and/or artificial sources.

As used herein, the term “isotope” refers to atoms of an element that have the same number of protons in their atomic nuclei but a different number of neutrons. Isotopes of a given element have the same atomic number but different mass numbers. Some isotopes may be stable while others are radioactive; furthermore, some isotopes may be more or less radioactive than other radioactive isotopes. Isotopes may be used in various applications, including but not limited to, mass spectrometry, nuclear magnetic resonance spectroscopy, and radioisotope labeling. In some cases, isotopes may be incorporated into chemical compounds, such as nucleotides or amino acids, to create isotopically labeled molecules for use in scientific research, medical diagnostics, or other fields.

As used herein, the term “mass spectrometry” or “MS” refers to an analytical technique used to measure the mass-to-charge ratio of ions. Mass spectrometry may be used to determine molecular mass, elemental composition, or structural information for a sample. Mass spectrometry includes, without limitation, liquid chromatography-mass spectrometry (LC-MS), tandem mass spectrometry (MS/MS), matrix-assisted laser desorption/ionization time-of-flight mass spectrometry (MALDI-TOF MS), and related techniques.

### System of Mass Tagged Polynucleotides

### Structure of the Mass Tagged Polynucleotides

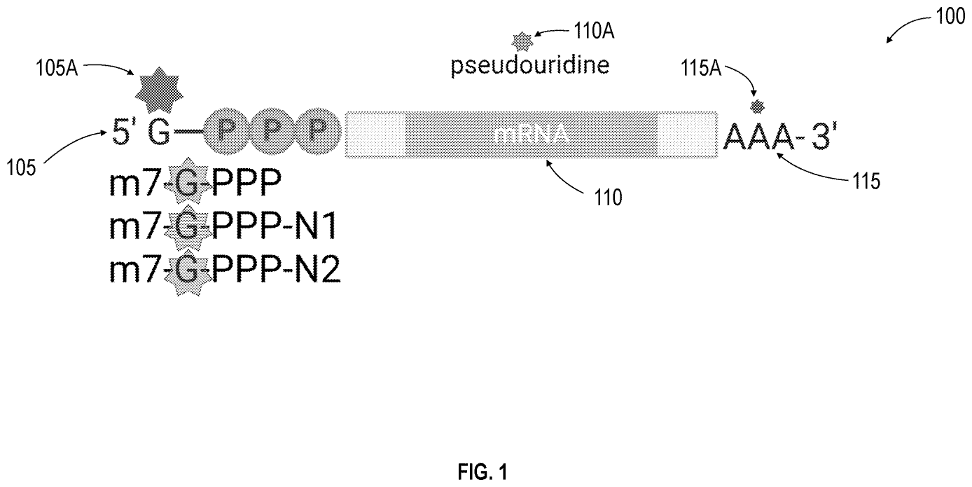

As illustrated in FIG. 1, the present disclosure describes an mRNA molecule 100 wherein one or more structural moieties of the polynucleotide 100 may comprise an isotopic ratio that differs from naturally occurring versions of the corresponding moiety. In a preferred embodiment, the polynucleotide can be an mRNA molecule suitable for research applications, therapeutic interventions, or vaccine development, such as mRNA encoding viral antigens for immunization purposes. The present disclosure provides that distinct moieties within the mRNA molecule 100 may each contain their own artificial isotopic ratios, which can permit characterization of the purity and efficiency of each step in industrial mRNA synthesis. In some instances, the mass-tagged nucleosides may be configured to serve as internal standards that facilitate quantitative analyses of oligonucleotide composition by mass spectrometry. The relative ratio between non-naturally occurring mass-tagged nucleosides can inform on the manufacturing product's integrity and purity, representing critical quality attributes (CQAs) that may be measured consistently throughout the life cycle of the RNA product. Alternatively, the isotopic labeling approach can provide a means for distinguishing synthetic nucleic acids from naturally occurring counterparts or from biological contaminants during quality assessment procedures.

In a preferred embodiment, the mRNA molecule 100 comprises an open reading frame 110, which may be translated in vitro or in vivo in order to generate a peptide or polypeptide of therapeutic or research interest. In another preferred embodiment, the mRNA molecule 100 may further comprise a 5′ guanosine cap 105, which can include an isotopically labeled guanosine 105A that serves as a mass-tagged capping agent. In some preferred embodiments, the mRNA molecule 100 may further comprise a poly-A tail 115 containing isotopically labeled adenosine 115A, wherein the percentage of mass-tagged adenosine in the poly-A tail 115 can be predetermined during synthesis. These structural components may be configured to enhance stability of the complete mRNA polynucleotide 100, in addition to facilitating translation initiation and protecting against exonuclease degradation. The present disclosure provides that each of these components can be synthesized in distinct stages utilizing appropriate reagents, wherein the open reading frame 110 may be capped with a 5′ guanosine cap 105 and then polyadenylated to generate a poly-A tail 115. In some instances, the percentage of mass-tagged nucleosides present in the RNA functional sequence can be controlled during the transcription process to achieve a desired isotopic ratio for quality control purposes.

In a preferred embodiment, the mRNA molecule 100 may be synthesized at different stages with different isotopically tagged polynucleotides to facilitate detection and characterization of low abundance synthetic contaminants and to provide improved means for benchmarking RNA products' quality assurance and quality control protocols. As illustrated in FIG. 1, the open reading frame 110 can be synthesized such that the isotopically tagged nucleotide comprises pseudouridine 110A, which may be incorporated at a predetermined ratio relative to unlabeled pseudouridine or uridine. In another preferred embodiment, the capping step may comprise isotopically tagged guanosine 105A and its derivations, wherein the mass-tagged capping agent can serve as an internal standard for assessing cap integrity throughout the product life cycle. The polyadenylation step can include isotopically tagged adenosine 115A at a controlled percentage within the poly-A tail 115, which may facilitate monitoring of tail length and integrity during storage and handling. In some preferred embodiments, upon enzymatic fragmentation and mass spectrometry analysis, the presence of isotopically tagged nucleosides from multiple structural regions within a single small fragment may indicate that an error has occurred during the manufacturing process, since these isotopic signatures can be expected to remain segregated in properly synthesized full-length mRNA molecules. Alternatively, a mixture of multiple highly similar mRNAs may be distinguished without expensive sequencing by synthesizing one variant with one isotopically labeled nucleotide and another variant with a different isotopically labeled nucleoside, such as distinguishing between mRNA molecules encoding different protein variants by incorporating isotopically labeled pseudouridine in one and isotopically labeled cytidine in another.

The present disclosure provides that libraries of stable isotopically labeled and modified nucleosides can enable quantitative analyses of oligonucleotide composition by mass spectrometry, thereby facilitating RNA product authentication and integrity validation. In a preferred embodiment, the astute introduction of mass-tagged nucleotides in RNA sequences, such as mRNA vaccines or miRNA therapeutics, may be configured to facilitate the detection and characterization of low abundance synthetic contaminants that might otherwise escape detection by conventional analytical methods. In some instances, the approach can generate tractable nucleic acids and provide access to certifiable reference materials that help assure measurement quality across different manufacturing batches and analytical laboratories. The relative ratio between the non-naturally occurring mass-tagged nucleosides in the 5′ guanosine cap 105, the open reading frame 110, and the poly-A tail 115 can inform on the manufacturing product's integrity and purity throughout the product life cycle. In another preferred embodiment, solvent-assisted mechanochemical approaches may be employed for the efficacious synthesis of isobaric isotopically labeled canonical and non-canonical nucleosides and nucleotides, which can then be incorporated into mRNA molecules during in vitro transcription. Alternatively, the mass-tagged nucleosides may optionally be used to explore and harness the complex field of epitranscriptomics and its therapeutic applications by providing improved means for RNA-omics analyses and RNA sequence product quality monitoring.

### Production of Mass Tagged Nucleosides

In a preferred embodiment, the present disclosure provides mixtures of isotopically labeled nucleosides that serve as valuable standards for LC-MSⁿ analyses, as the individual components can be detected and quantified separately within complex samples. Nucleosides incorporating ¹³C- or ¹⁸O atoms may be characterized as isotopologs that retain the chromatographic properties of their naturally occurring nucleoside counterparts, properties that can be leveraged to study nucleotide turnover and quantify low abundance nucleotides in RNA products. The exchange of a 160 with an 180 in a nucleoside changes its exact isotopic mass by approximately 2.0042 Da, which can provide a detectable mass shift without altering the chemical behavior of the molecule. This mass change may be significant and can be readily detected by LC-MS since the combined natural abundance of a mass (m)+2 nucleoside is typically less than 1.5% compared with the monoisotopic nucleoside. High-resolution MS instruments, such as the Q-Exactive LC-MS, can differentiate between each isotopolog, particularly when combined with targeted MS approaches including multiple reaction monitoring (MRM) or selected reaction monitoring (SRM). In some instances, the detection of the chemical isotopolog may facilitate the identification of coeluted parent species, while MS² fragmentation measurements can confirm the nucleoside's identity and provide structural information.

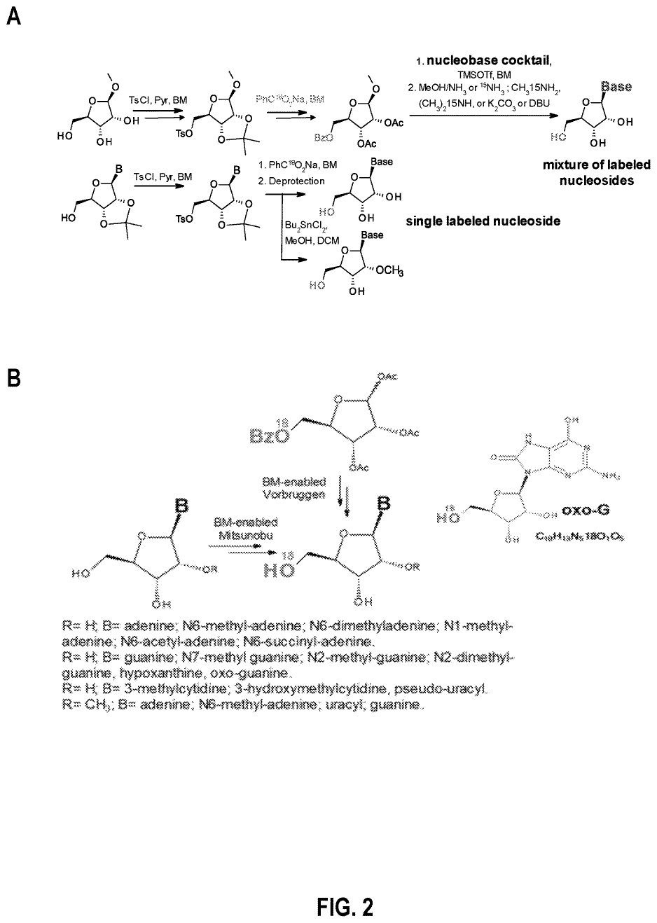

As illustrated in FIG. 2, the present disclosure provides solvent-assisted mechanochemical approaches to the efficacious synthesis of nucleoside isotopologs that can enable cost-effective production of mass-tagged building blocks for RNA quality assurance and quality control applications. In a preferred embodiment, the nucleoside isotopologs may be generated via a facile isotopic exchange of the 5′-oxygen of ribosides, a process that can be applied to many commercially available and non-commercially available nucleosides. Taking adenosine (C₁₀H₁₃N₅O₄) as an example, the (m and m+2) isotopic masses may be reported at 267.09678 (100%), 269.09718 (0.2%), 269.10102 (0.8%) and 269.10349 (0.5%), while the exact mass for a singly ¹⁸O modified adenosine (C₁₀H₁₃N₅¹⁸O₁O₃) can be 269.10000 (100%). While the cost of isotopically labeled raw materials may be substantially higher than naturally occurring entities, incorporation of a stable isotope into a nucleoside scaffold can have a relatively low impact on overall production cost when the incorporation process achieves high yields. The relative peak intensity of the nucleoside compared to that of the isotopolog can inform on its abundance, thereby providing quantitative data for quality control assessments of RNA products including mRNA vaccines and miRNA therapeutics.

In another preferred embodiment, mixtures of nucleosides from a mixture of nucleobases may be produced in high yields using a one-pot, two-step mechanical Vorbrüggen conversion, as illustrated in FIG. 2. This multi-nucleobase approach can be applied to a C5′-¹⁸O-labeled riboside ester to generate C5′-¹⁸O-labeled nucleoside libraries that can serve as certifiable reference materials for RNA-omics analyses. In some preferred embodiments, the approach may be applied to triacetoxyacetate-2′-methyl-C5′-¹⁸O-labeled riboside to generate C5′-¹⁸O-2′-methylnucleosides, which can be incorporated into synthetic RNA molecules for quality monitoring purposes. The present disclosure contemplates that nucleobases of similar reactivity may optionally be parametrized using machine-learning algorithms to optimize reaction conditions that achieve favorable product distributions and yields. In some instances, the mechanochemical synthesis approach may be configured to produce both canonical nucleosides such as adenosine, guanosine, cytidine, and uridine, as well as non-canonical and modified nucleosides including pseudouridine, N6-methyladenosine, and 8-oxoguanosine. Alternatively, the synthesis protocols can be adapted to generate libraries of isotopically labeled nucleosides bearing 15N labels in the nucleobase portion, which may provide complementary mass signatures for multi-dimensional quality control analyses.

In some preferred embodiments, the present disclosure provides for the astute introduction of mass-tagged nucleotides at specific positions within RNA sequences to facilitate the detection and characterization of low abundance synthetic contaminants. As illustrated in FIG. 1, a synthetic mRNA molecule 100 may be configured to include a mass-tagged capping agent comprising an isotopically labeled guanosine 105A in the 5′ guanosine cap 105, a percentage of mass-tagged nucleosides such as pseudouridine 110A in the open reading frame 110, and a percentage of mass-tagged adenosine 115A in the poly-A tail 115. The relative ratio between these non-naturally occurring mass-tagged nucleosides can inform on the manufacturing product's integrity and purity, representing critical quality attributes (CQAs) that may be measured consistently throughout the life cycle of the RNA product. In some instances, the mass-tagged nucleosides may be incorporated at ratios between 1:10 and 1:1000 relative to their unlabeled counterparts, depending on the sensitivity requirements of the analytical method employed. This approach can generate tractable nucleic acids and provide access to certifiable reference materials that help assure measurement quality for RNA products including mRNA vaccines and therapeutic oligonucleotides. Alternatively, different isotopic labels may be employed at each structural region of the mRNA molecule to enable simultaneous monitoring of multiple quality parameters in a single LC-MS analysis.

As illustrated in FIG. 2, the present disclosure provides two routes by which isotopically labeled nucleosides can be synthesized from commercially available precursors using solvent-assisted mechanochemical approaches. In a preferred embodiment, isotopically M+2 labeled nucleosides can be produced from the nucleoside by nucleophilic displacement of ¹⁶O at the C5′ position, thereby introducing an ¹⁸O label that provides a detectable mass shift for quality control applications. The acylated ¹⁸O-riboside generated from the commercially available 1-O-methyl, 2,3-isopropylidene riboside may be used to produce individual ¹⁸O-nucleosides or mixtures of labeled nucleosides by ball milling-enabled Vorbrüggen coupling reactions. In some instances, this approach can generate libraries of stable isotopically labeled nucleosides that enable quantitative analyses of oligonucleotide composition by mass spectrometry. The mechanochemical synthesis may be configured to produce both canonical nucleosides such as adenosine, guanosine, cytidine, and uridine, as well as non-canonical nucleosides including pseudouridine and N1-methylpseudouridine. Alternatively, the synthesis can be adapted to generate modified nucleosides such as 8-oxoguanosine, which may serve as internal standards for detecting oxidative damage in RNA products during quality assurance protocols.

In another preferred embodiment, the isolated yield of S_{N}2 reactions at the C5′ position of individual nucleosides may depend on the reaction conditions, including the specific nucleoside substrate, solvent selection, temperature parameters, and the mechanochemical apparatus employed. The yields of glycosylation reactions can similarly rely on the nucleobase identity, the potential need for activation or protection of the nucleobase, and the nature of the riboside precursor. In some preferred embodiments, the scope and limiting parameters of direct ¹⁸O incorporation at C5′ of nucleosides or C5 of ribosides may be optimized during the process of scaling the reaction for mass production to support industrial-level RNA manufacturing. The present disclosure contemplates that to access a library of ¹⁸O, ¹⁵N-containing isotopologs, 6-chloro-purine and 6-hydroxy-2-chloro-purine nucleosides can be prepared by Vorbrüggen chemistry according to processes attested in the prior art, followed by methanolic lysine deacylation, and subsequently by chloride displacement with ¹⁵N-ammonia or (di)methyl-¹⁵N-amine in ethanol to gain access to the ¹⁵N-modified purine nucleoside series. In some instances, based on cost considerations and availability, the nucleosides may be synthesized de novo or commercially available purine and pyrimidine nucleobases and nucleosides may be acquired as starting materials. The mechanochemistry-enabled conditions may then generate m+2 nucleosides, such as ¹⁸O-oxo-guanosine as illustrated in FIG. 2A, which can serve as tracers for monitoring RNA product integrity throughout the manufacturing lifecycle.

In a preferred embodiment, the ease of reaction monitoring afforded by mechanochemical synthesis may be harnessed to parametrize rates and yields to reaction conditions for each target molecule, thereby facilitating optimization of isotopically labeled nucleoside production. The present disclosure contemplates that astute introduction of mass-tagged nucleotides in RNA sequences, such as mRNA vaccines or miRNA therapeutics, can facilitate the detection and characterization of low abundance synthetic contaminants. In some preferred embodiments, the relative ratio between non-naturally occurring mass-tagged nucleosides may inform on the manufacturing product's integrity and purity, representing a critical quality attribute that can be measured consistently throughout the lifecycle of the RNA product. The mass-tagged capping agent, which may be configured to comprise an isotopically labeled guanosine, can provide a signature for authenticating the 5′ terminus of synthetic mRNA molecules. Alternatively, a defined percentage of mass-tagged nucleosides present in the RNA functional sequence may serve as internal standards for quantifying the coding region integrity. The percentage of mass-tagged adenosine in the poly-A tail can optionally provide an additional marker for assessing 3′ terminal integrity, and the combined analysis of these three regions may enable comprehensive quality control assessment of synthetic RNA products.

### Production of Mass-Tagged M7-Guanosine for Capping

The present disclosure addresses the challenges associated with monitoring m7-guanosine in RNA-based biotechnologies, where this modified nucleobase serves as a component of the 5′-terminus capping structure that can facilitate effective translation. As illustrated in FIG. 1, the 5′ guanosine cap 105 containing isotopically labeled guanosine 105A may be incorporated into the mRNA molecule 100 to enable improved detection and quantification during quality control procedures. In a preferred embodiment, m7-guanosine triphosphate can modify the 5′-terminus nucleotide of RNA, and together with the 3′-poly-A tail 115, the m7-GpppN-5′-capping may help stabilize RNA constructs for applications such as mRNA vaccines or guide RNA therapeutics. The retention of this modified nucleobase in the RNA product can serve as a quality attribute for RNA products intended for gene editing or translation applications. However, monitoring this modified nucleobase presents challenges because transcriptomic analyses typically validate its presence indirectly, which may be potentially misleading depending on the efficiency with which a sequence is transcribed. The present disclosure provides approaches that may address these monitoring challenges through the use of isotopically labeled nucleosides that can serve as internal standards for mass spectrometry-based analyses.

In some preferred embodiments, the detection of m7-guanosine by LC-MS can be challenging because this modified base may be easily lost in the analytical noise, particularly when it represents a single methylated guanosine among many nucleotides in a given sequence. The methyl group can also be lost during sample processing, which may further complicate accurate quantification of this modified nucleobase. To address these analytical challenges, the present disclosure provides access to isotopically labeled m7-guanosine that may serve as internal standards for LC-MS² analyses and as precursors to isotopically labeled m7-GpppN capping agents. These isotopically labeled compounds can be highly valuable when using LC-MS² to conduct global analyses of the nucleosidic composition of an RNA product. In some instances, the mass-tagged capping agent may be configured to provide a non-naturally occurring isotopic signature that can distinguish synthetic RNA products from naturally occurring RNA or contamination. The relative ratio between non-naturally occurring mass-tagged nucleosides can inform on the manufacturing product's integrity and purity throughout the life cycle of the RNA product.

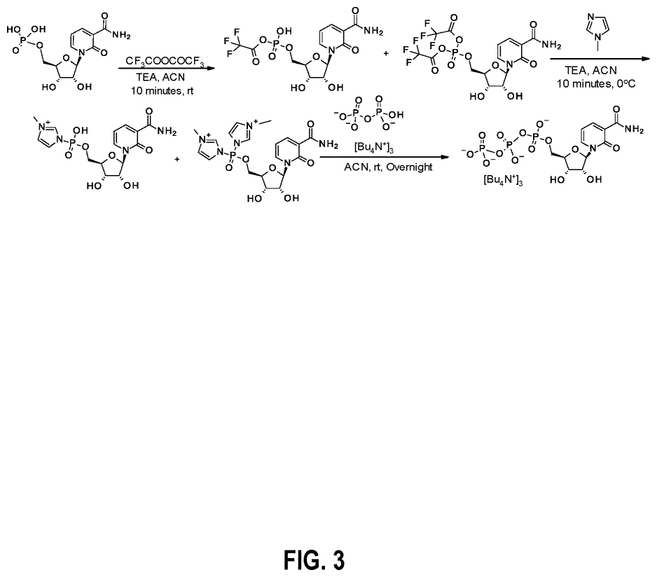

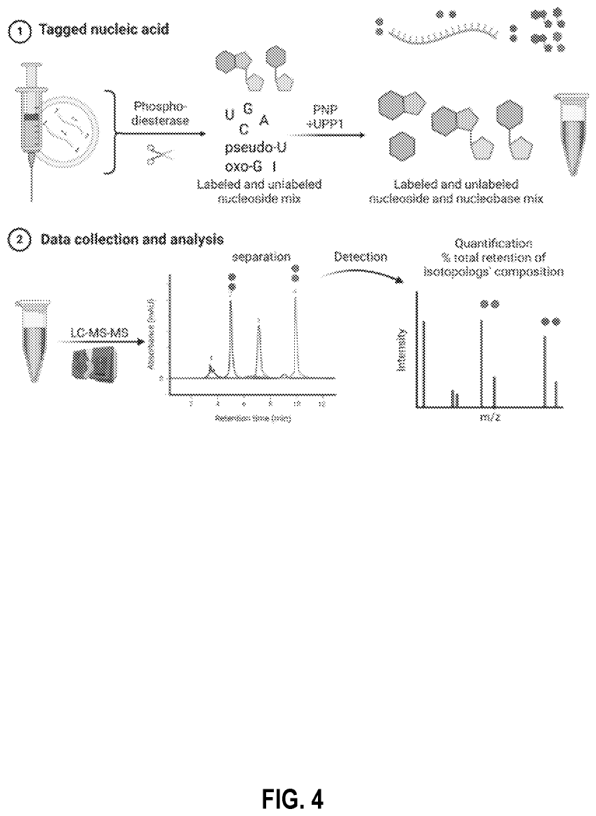

In a preferred embodiment, ¹⁸O-m7-guanosine may be generated from ¹⁸O-guanosine for the synthesis of ¹⁸O-m7-GpppN by methylation using dimethylsulfate, as illustrated in FIG. 2 and FIG. 3. Alternatively, ¹⁸O, ¹³C-methyl-7-guanosine can be synthesized using ¹³C-dimethylsulfate to introduce both oxygen and carbon isotopic labels into the modified nucleoside. In another preferred embodiment, the doubly labeled ¹⁸O, ¹³C-methyl-7-guanosine may be utilized as an internal standard for LC-MS analyses, where it can optionally reveal degradation by demethylation of ¹⁸O-m7-guanosine during sample processing when spiked into the sample to be analyzed. This approach may be configured to provide quality assurance and quality control capabilities that can be measured consistently throughout the life cycle of the RNA product. As illustrated in FIG. 4, the analytical workflow can enable detection and quantification of isotopically labeled nucleosides following enzymatic digestion and LC-MS analysis. In some instances, the use of multiple isotopic labels may provide additional information regarding sample integrity and processing conditions that can inform quality control decisions.

### Synthetic Scalability by Mechanochemical Means

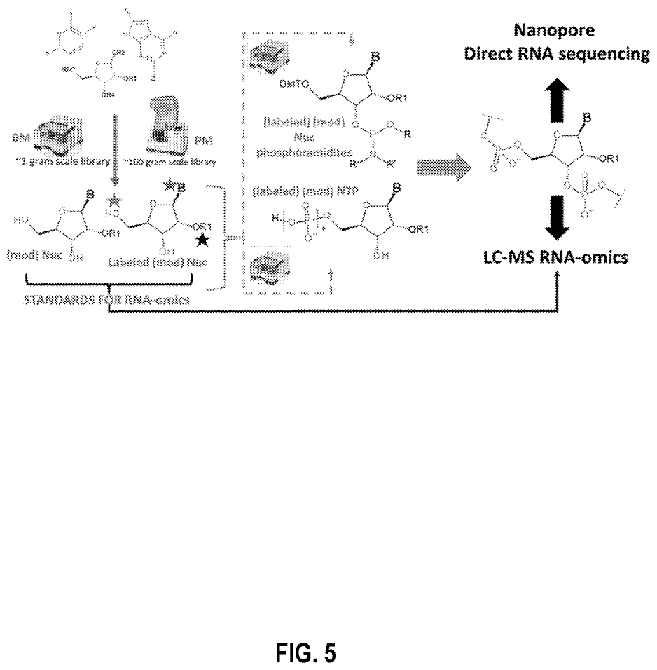

Ribose tetraacetate and nucleobases are particularly amenable to Vorbrüggen reaction conditions under milling conditions, with almost 100% conversion of the nucleobase to the fully protected nucleosides. In a preferred embodiment, the conditions may be optimized through informed iteration via machine learning algorithms to improve the model's overall predictive capacity upon profiling the parameters reporting the reaction outcomes vs. conditions (i.e., amount of remaining starting material, product, and byproducts vs. molecular solvents, reagents' type and mole equivalency, BM-frequency in Hz and time, homogenizer reaction time, temperature, pressure). The present disclosure contemplates that conditions' variation testing, outcomes' deviation, and resilience of the optimized conditions to variation parameters can be investigated to refine the synthetic protocols. In some instances, the machine learning approach may be configured to identify correlations between reaction parameters that would not be apparent through conventional experimental design methodologies. Once derived, the best conditions can then be used to generate batches of 1-10 gms of ¹⁸O—C₅′-adenosine, ¹⁸O—C₅′-guanosine, ¹⁸O—C₅′-8-oxo-guanosine, ¹⁸O—C₅′-cytidine, and ¹⁸O—C₅′-uridine, as well as the 2′-O and N-¹³C-methyl derivatives. Alternatively, larger scale production may be achieved using planetary milling apparatus, as illustrated in FIG. 5, which can accommodate approximately 100-gram scale library production for industrial applications.

The addition of an isotopically labeled nucleoside to a sample as an internal standard in LC-MS measurements allows for the simultaneous detection and quantification of the given nucleoside in a complex mixture. The absolute quantification of multiple nucleosides can be achieved simultaneously using mixtures of isotopically labeled nucleosides of known mole composition. In a preferred embodiment, cocktails of ¹³C₅- or ¹⁸O-ribose-containing nucleosides (M+5 and M+2, respectively) may be generated by applying mechanochemical conditions to multiple purine and pyrimidine nucleobases. In another preferred embodiment, mixtures of the ¹³C₅ or 5′-¹⁸O-labeled 1-N-Me, 6-N-Me, and 6-N-Me₂ adenosine and guanosine can be generated from the respective chloropurine ribosides using methylamine and dimethylamine in methanol under sealed pressure tube conditions. The optimized (machine-learned) reaction profile gathered for each nucleoside may be tabulated to achieve the highest conversions within the mixtures, and nucleosides generated under similar conditions can be matched for multicomponent reaction optimizations. As such, machine learning applied to condition optimization helps to refine the reaction design to deliver the best protocol. In some instances, the resulting isotopically labeled nucleoside libraries may serve as certifiable reference materials that help assure measurement quality across different analytical platforms and laboratories.

The present disclosure provides innovative technologies that may improve the protocols supporting RNA products' quality assurance (QA) and quality control (QC), which can be particularly valuable for exploring and harnessing the complex field of epitranscriptomics and its therapeutic applications. In some preferred embodiments, the solvent-assisted mechanochemical approaches described herein may be configured to enable efficacious syntheses of nucleosides, nucleotides, and dinucleotides as well as short oligonucleotides. This knowhow can be applied to the production of isobaric isotopically labeled, canonical and non-canonical as well as modified nucleosides and nucleotides for various analytical applications. As illustrated in FIG. 1, the mRNA molecule 100 may incorporate isotopically labeled nucleosides at specific structural regions including the 5′ guanosine cap 105, the open reading frame 110, and the poly-A tail 115. The isotopically labeled guanosine 105A in the 5′ guanosine cap 105 can serve as a mass-tagged capping agent, while the pseudouridine 110A in the open reading frame 110 and the isotopically labeled adenosine 115A in the poly-A tail 115 provide additional mass signatures for comprehensive product characterization. In some instances, the relative ratio between these non-naturally occurring mass-tagged nucleosides may inform on the manufacturing product's integrity and purity, representing critical quality attributes (CQAs) that can be measured in the same manner throughout the life cycle of the RNA product.

To facilitate RNA product authentication and integrity, the present disclosure contemplates that libraries of stable isotopically labeled and/or modified nucleosides can enable quantitative analyses of oligonucleotides' composition by mass spectrometry. In a preferred embodiment, astute introduction of mass-tagged nucleotides in RNA sequences, such as mRNA vaccines or miRNA therapeutics, may facilitate the detection and characterization of low abundance synthetic contaminants. As illustrated in FIG. 4, the analytical workflow can process tagged nucleic acids through enzymatic digestion with phosphodiesterase to release constituent nucleosides, followed by treatment with PNP and UPP1 enzymes to generate a labeled and unlabeled nucleoside and nucleobase mix. The LC-MS-MS analysis may then be configured to separate, detect, and quantify the isotopic species present in the sample. In some preferred embodiments, this approach provides improved means for the benchmarking of RNA products' QA/QC protocols for RNA-omics analyses and RNA sequence product quality and integrity monitoring. Alternatively, the workflow may be adapted to accommodate different sample types or analytical requirements depending on the specific application.

In another preferred embodiment, the approach described herein may generate tractable nucleic acids and provide access to certifiable reference materials that help assure measurement quality across different manufacturing sites and analytical laboratories. The introduction of a mass-tagged capping agent based on guanosine, combined with a defined percentage of mass-tagged nucleosides present in the RNA functional sequence and a percentage of mass-tagged adenosine in the poly-A tail, can create a unique isotopic signature for each synthetic RNA product. As illustrated in FIG. 5, the synthesis and analysis workflow may incorporate labeled and modified nucleoside phosphoramidites and labeled and modified nucleoside triphosphates as building blocks for RNA synthesis. In some instances, the synthesized RNA products can be analyzed by Nanopore Direct RNA sequencing or LC-MS RNA-omics analysis to verify isotopic label incorporation and assess product integrity. The present disclosure contemplates that this dual analytical approach may provide complementary information about the mass-tagged RNA molecules. Optionally, additional analytical techniques may be employed in conjunction with these methods to provide further characterization of the synthetic RNA products.

The present disclosure provides methods that may be configured to monitor the relative abundance of isotopically labeled nucleosides throughout the manufacturing process and during storage of the RNA product. In a preferred embodiment, deviations from the expected isotopic ratios can indicate the presence of contamination from biological sources, degradation of the RNA product, or other quality issues that may affect product performance. For instance, a decrease in the relative abundance of isotopically labeled adenosine 115A in the poly-A tail 115 compared to the isotopically labeled guanosine 105A in the 5′ guanosine cap 105 may suggest preferential degradation of the 3′ terminus of the mRNA molecule 100. In some preferred embodiments, the isotopic signature can serve as an authentication marker that distinguishes the synthetic RNA product from naturally occurring RNA or from products manufactured by different processes. Alternatively, the isotopic labeling pattern may be customized for different product batches or manufacturing sites to enable traceability throughout the supply chain. In some instances, the mass spectrometry data may be compared to internal standards comprising data regarding the relative abundance of the isotopically labeled nucleosides compared with their unlabeled counterparts during the synthesis of the synthetic mRNA molecule, as described in the quality control methods of the present disclosure.

### Generation of Isotopically Labeled Nucleotides and Nucleoside Triphosphates

In a preferred embodiment, the present disclosure addresses the challenge of producing isotopically labeled nucleotide triphosphates at financially feasible prices for incorporation into RNA oligomers. Although several modified nucleotide triphosphates are commercially available from suppliers, these reagents are typically very expensive and may limit widespread adoption for quality assurance and quality control applications. The present disclosure provides low-cost syntheses of isotopically labeled nucleoside mono- and diphosphates (NMPs, NDPs) by direct milling of isotopically labeled nucleosides using POCl₃ under mechanochemical conditions. In some preferred embodiments, isotopically labeled nucleoside triphosphates (NTPs) can be generated via a methyl-imidazolate enabled conversion of NMP to NTP, which may be readily applicable to the production of RNA capping reagents such as m7-GpppN. This approach can facilitate the generation of mass-tagged capping agents, mass-tagged nucleosides for incorporation into functional RNA sequences, and mass-tagged adenosine for poly-A tail synthesis. The relative ratio between these non-naturally occurring mass-tagged nucleosides may inform on the manufacturing product's integrity and purity throughout the life cycle of the RNA product.

As illustrated in FIG. 3, modified nucleoside triphosphates can be generated directly from the corresponding nucleoside through a multi-step process. In a preferred embodiment, the POCl₃-conversion of isotopically labeled nucleosides may be readily achieved on a wide range of unprotected nucleosides on a multigram scale by milling to access isotopically labeled NTPs. Exemplary isotopically labeled nucleosides that can be processed through this approach include ¹⁸O-pseudouridine, ¹⁸O—N1-methyl-pseudouridine, ¹⁸O-adenosine, ¹⁸O-oxo-guanosine, and ¹⁸O-m7-guanosine. In some instances, labeled NTPs generated through this approach can be used to mass-tag RNA products, thereby enabling detection and characterization of low abundance synthetic contaminants in mRNA vaccines or miRNA therapeutics. Alternatively, these labeled NTPs may serve as internal standards for LC-MS absolute quantification of NMP, NDP, and NTP concentrations. The astute introduction of mass-tagged nucleotides in RNA sequences can provide improved means for benchmarking RNA products' QA/QC protocols and monitoring RNA sequence product quality and integrity.

In another preferred embodiment, the isotopically labeled canonical and N-Me, N-Me₂, and 2′-OMe-nucleoside triphosphates may be generated along with the 8-hydroxy-guanosine, inosine, and (N1-methyl-) pseudouridine triphosphates from the corresponding nucleosides. As illustrated in FIG. 2 and FIG. 3, the synthesis can proceed from commercially available precursors through mechanochemical approaches that have been developed by the laboratory. The conversion of the nucleoside to a dichlorophosphate ester by ball milling (BM) represents a central transformation in this chemistry. The dichlorophosphate intermediate may be hydrolyzed to the phosphate after removal of the unreacted POCl₃ by trituration with anhydrous ether or alternatively converted to the triphosphate via the methyl-imidazolate pathway. In some preferred embodiments, each of these materials may be generated in sufficient amounts (greater than 1 mmol) to be tested as substrates of RNAP in vitro assays and used as standards in LC-MS method development. This approach can generate tractable nucleic acids and provide access to certifiable reference materials that help assure measurement quality for epitranscriptomics and therapeutic applications.

In some instances, certain limiting factors may affect high yields, and in a preferred embodiment, these factors can be avoided or mitigated to ensure optimum production of the desired reagent. For instance, pretreatment of the acetonitrile solution of the butylammonium pyrophosphate salts with 3 Å molecular sieves can dramatically reduce the water content of the reaction solution, as measured by ¹H NMR, and may increase yields. Accordingly, a preferred embodiment may be configured to introduce molecular sieves in the anhydrous acetonitrile in each reaction protocol as part of standard operating procedure. Once this has been addressed, the chemistry can be conducted on a 0.1 mmol scale by BM (approximately 25-30 mg modified nucleoside per reaction) on individual nucleosides. In another preferred embodiment, once optimized, the reaction may be performed on a greater than 1 mmol scale (greater than 250-300 mg) to demonstrate scalability and identify potential issues linked to water content upon scale-up. Crude materials can optionally be purified by C18-reversed phase chromatography to minimize hydrolysis and salt contamination, and each entity may be fully characterized before being used as an LC-MS standard. The resulting mixtures of isotopically labeled nucleotides and nucleoside triphosphates are poised for use as internal standards for LC-MS analyses, enabling quantitative analyses of oligonucleotides' composition by mass spectrometry.

In some preferred embodiments, the aforementioned mixtures of isotopically labeled nucleosides may be phosphorylated and converted to the triphosphates according to this protocol at approximately 0.1-1 mmol scale. The present disclosure contemplates application of this chemistry to the aforementioned isotopically labeled oxo-guanosine, pseudouridine, inosine, and the modified O- and N-methylated nucleosides in addition to commercially available nucleosides such as N-Me-adenosine. Libraries of stable isotopically labeled and modified nucleosides generated through these approaches can enable quantitative analyses of oligonucleotides' composition by mass spectrometry for RNA product authentication and integrity verification. In some instances, the innovation of introducing a mass-tagged capping agent (guanosine-based), a percentage of mass-tagged nucleoside present in the RNA functional sequence, and a percentage of mass-tagged adenosine in the poly-A tail may be configured to provide comprehensive quality metrics. The relative ratio between the non-naturally occurring mass-tagged nucleosides can inform on the manufacturing product's integrity and purity as a critical quality attribute (CQA) that may be measured in the same manner throughout the life cycle of the RNA product. This approach can facilitate the detection and characterization of low abundance synthetic contaminants and provide improved means for benchmarking RNA products' QA/QC protocols for RNA-omics analyses.

### Synthesis of Methylated Guanosine Dinucleotides

In a preferred embodiment, the present disclosure addresses the production of effective capping agents at the 5′-end of RNA sequences, which can serve as master regulators of translation initiation and RNA polymerization. The most common capping agent employed in synthetic RNA production may be m7GpppG, which comprises N7-methylated guanosine triphosphate linked to 5′-guanosine through a triphosphate bridge. In some instances, derivatives of m7GpppN can alternatively be utilized as capping agents, including those incorporating a 2′-methyl modification (designated as cap 1) or a 3′-nucleotide extension (designated as cap 2 and higher order cap structures). The generation of these capping nucleotides presents technical challenges, particularly with respect to the formation of the triphosphate linkage that connects the methylated guanosine to the adjacent nucleoside. While enzymatic processes have been developed for cap synthesis, these approaches may remain limited in their scope and scalability for industrial applications. The imidazolide route, though chemically feasible, can present difficulties when attempting to scale up production to quantities suitable for commercial RNA manufacturing operations.

In some preferred embodiments, the present disclosure provides mechanochemical approaches that may be configured to generate triphosphate linkages through displacement of a 5′-tosyl group on a given nucleoside with tetrabutylammonium nucleoside triphosphate (Bu₄N)₄-NTP under ball milling conditions. This solvent-assisted mechanochemical approach can enable the efficacious synthesis of capping agents at both milligram and gram scales, starting with [4(Bu₄N)]-m7GTP and 5′-tosyl-guanosine as starting materials. In another preferred embodiment, after reaction modeling and optimization, the synthesis may be performed with isotopically labeled [4(Bu₄N)]-¹⁸O-m7GTP to generate mass-tagged capping agents suitable for quality assurance and quality control applications. As illustrated in FIG. 1, the isotopically labeled guanosine 105A incorporated into the 5′ guanosine cap 105 can provide a detectable mass signature that facilitates authentication and integrity monitoring of synthetic mRNA products throughout their lifecycle. The introduction of mass-tagged capping agents, combined with a defined percentage of mass-tagged nucleosides in the RNA functional sequence and mass-tagged adenosine in the poly-A tail 115, can enable quantitative assessment of manufacturing product integrity and purity through mass spectrometry analysis.

In some preferred embodiments, the mechanochemical parameters optimized for Cap 0 synthesis may be applied to generate isotopically labeled Cap 1 structures from 5′-tosyl-2′-O-methyl N1-methyladenosine and Cap 2 structures from 5′-tosyl-2′-O-methyl N1-methyladenosine-3′-5′-guanosine dinucleotide precursors. These higher-order cap structures can optionally incorporate isotopic labels at specific positions to provide distinct mass signatures detectable by LC-MS or MALDI-TOF MS analytical methods. In some instances, the relative ratio between non-naturally occurring mass-tagged nucleosides in the cap structure, the open reading frame 110, and the poly-A tail 115 may inform on critical quality attributes that can be measured consistently throughout the lifecycle of the RNA product. As illustrated in FIG. 5, libraries of stable isotopically labeled and modified nucleosides generated through mechanochemical synthesis can enable quantitative analyses of oligonucleotide composition and facilitate the detection of low-abundance synthetic contaminants. The present disclosure thus provides access to certifiable reference materials that may help assure measurement quality in RNA-omics analyses and support benchmarking of quality assurance and quality control protocols. Alternatively, different isotopic labels such as ¹³C or ¹⁵N may be incorporated into the capping agents to provide complementary mass signatures for multiplexed quality control applications.

Synthesis of Isotopically Labeled Nucleoside-Containing Oligonucleotides from Isotopically Labeled Nucleoside Phosporamidites

The present disclosure recognizes that not all modified nucleoside triphosphates serve as suitable substrates for RNA polymerase enzymes, and not all RNA oligomers can be accessed through conventional RNA transcription methodologies. In some instances, chemical synthesis may be employed to access short RNAs or oligonucleotides that incorporate specific modifications or multiple modifications at defined positions within the sequence. Short oligomers can be synthesized chemically from nucleoside phosphoramidites using solid phase or liquid phase chemistry approaches, with each approach offering distinct advantages depending on the target sequence and modification pattern. While phosphoramidites are commercially available for canonical nucleosides such as adenosine, guanosine, cytidine, and uridine, isotopically labeled versions of these building blocks remain commercially unavailable. The mechanochemical processes described herein can be as efficient on P(III) reagents, such as chlorophosphoramidites and phosphorodiamidites, as they are on P(V) reagents, including chlorophosphates and chlorophosphoramidates. Accordingly, mechanochemistry may be applied to generate several commercially unavailable isotopically labeled nucleoside phosphoramidites that can be used to synthesize specific short mass-tagged sequences for quality assurance and quality control applications.

In a preferred embodiment, isotopically labeled nucleoside phosphoramidites may be configured to serve as internal standards for MALDI-MS² analyses, such as ToF/TOF methodologies, enabling detection and characterization of minor contaminants including oxidized guanine species in synthetic sequences. The astute introduction of mass-tagged nucleotides in RNA sequences, such as mRNA vaccines or miRNA therapeutics, can facilitate the detection and characterization of low abundance synthetic contaminants that may otherwise escape detection by conventional analytical methods. In some preferred embodiments, the relative ratio between non-naturally occurring mass-tagged nucleosides may inform on the manufacturing product's integrity and purity, representing critical quality attributes that can be measured consistently throughout the life cycle of the RNA product. As illustrated in FIG. 1, the mRNA molecule 100 may incorporate isotopically labeled guanosine 105A in the 5′ guanosine cap 105, pseudouridine 110A in the open reading frame 110, and isotopically labeled adenosine 115A in the poly-A tail 115, thereby providing multiple reference points for quality assessment. The innovation of this approach includes the introduction of a mass-tagged capping agent based on guanosine, a percentage of mass-tagged nucleosides present in the RNA functional sequence, and a percentage of mass-tagged adenosine in the poly-A tail. These mass-tagged building blocks can provide access to certifiable reference materials that help assure measurement quality across different analytical platforms and manufacturing facilities.

In another preferred embodiment, the cyanodiisopropyl phosphoramidites of canonical labeled nucleosides, pseudouridine, N-methylated pseudouridine, and oxoguanosine may be generated by mechanochemical means using ball milling or planetary milling apparatus. Different molar excess conditions can be tested during the synthesis, and the reactions may be monitored by in situ ³¹P NMR spectroscopy, with chemical shift changes from approximately 190 ppm for chlorophosphoramidite starting materials to approximately 140 ppm for the phosphoramidite products indicating reaction progress. In some instances, machine learning algorithms based on reaction outcome data may be employed to optimize the synthesis conditions and improve yields of the desired phosphoramidite products. The resulting nucleoside phosphoramidites can then be purified by C18-column chromatography or other suitable purification techniques to achieve the purity levels appropriate for oligonucleotide synthesis. Once fully characterized by analytical methods including NMR spectroscopy and mass spectrometry, the phosphoramidites may be used to synthesize RNA oligomers of approximately ten nucleotides in length possessing various combinations of labeled adenosine, guanosine, cytidine, uridine, oxo-guanosine, and pseudouridine. As illustrated in FIG. 5, the workflow for generating these building blocks encompasses mechanochemical synthesis at different scales, conversion to phosphoramidites or triphosphates, and subsequent incorporation into RNA molecules for quality control applications.

The present disclosure provides innovative technologies that can improve the protocols supporting RNA products' quality assurance and quality control, enabling exploration and harnessing of the complex field of epitranscriptomics and its therapeutic applications. In some preferred embodiments, libraries of stable isotopically labeled and modified nucleosides may enable quantitative analyses of oligonucleotide composition by mass spectrometry, providing benchmarking capabilities for RNA-omics analyses and RNA sequence product quality monitoring. Alternatively, the isotopically labeled nucleoside triphosphates and phosphoramidites generated through mechanochemical approaches can be used to prepare mass-tagged mRNA and oligonucleotides for quality control processes across different manufacturing environments. The solvent-assisted mechanochemical approaches developed for the efficacious synthesis of nucleosides, nucleotides, and dinucleotides as well as short oligonucleotides may be applied to the production of isobaric isotopically labeled canonical, non-canonical, and modified nucleosides and nucleotides. In some instances, these mass-tagged building blocks can facilitate RNA product authentication and integrity verification by providing tractable nucleic acids with defined isotopic signatures. The approach described herein may optionally generate certifiable reference materials that help assure measurement quality and enable consistent quality assessment throughout the manufacturing and storage lifecycle of RNA therapeutic products.

Method of Testing mRNA Purity With Mass Tagged Polynucleotides

In a preferred embodiment, the present disclosure provides methods for introducing stable isotopes into the sugar moiety of nucleosides of interest, which can enable determination of the relative level of labeled nucleotide incorporation into a given RNA sequence. The incorporation of isotopically labeled nucleosides may facilitate detection of low occurrence modifications, such as oxidation of guanine to form 8-oxoguanosine, through global mass spectrometry analyses. As illustrated in FIG. 4, LC-MS-based analytical workflows may be configured to process tagged nucleic acids and quantify the relative abundance of isotopically labeled species compared to their unlabeled counterparts. In some instances, MS-analyses can reveal the location of mass-tagged nucleotides within the RNA sequence, providing positional information that may be valuable for quality control applications. Isobaric isotopologs, such as 5′-¹⁸O-nucleosides, can allow for comprehensive characterization of synthetic RNA products by generating distinct mass signatures that are distinguishable from naturally occurring isotopic distributions. The present disclosure contemplates that such isotopic labeling approaches may enable improved quality assurance and quality control protocols for RNA-based therapeutics, including mRNA vaccines and miRNA therapeutics.

In some preferred embodiments, the natural abundance of M+2 mass in nucleosides, nucleotides, and nucleoside triphosphates may be approximately 0.8%, 1.5%, and 2.5%, respectively, which provides a baseline against which isotopically labeled species can be detected. The incorporation of one ¹⁸O-labeled nucleoside at 100% isotopic abundance within an RNA sequence of approximately 100 nucleotides containing naturally occurring isotopes may therefore be detectable by global LC-MS based RNA-omics, as the M+2 signal can shift from approximately 0.8% to approximately 2%. Similarly, incorporation of ¹⁸O-labeled nucleosides in triphosphate pools may be detectable as the M+2 signal can shift from approximately 2.5% to approximately 4.5%. In another preferred embodiment, global LC-MS tracing can be achieved with ¹⁸O-riboside-modified nucleosides to monitor RNA products' quality throughout the manufacturing lifecycle. The present disclosure contemplates that such mass-tagged nucleosides may serve as internal standards for quantitative assessment of RNA product integrity and purity. Alternatively, other isotopic labels such as ¹³C or ¹⁵N may be employed to generate different mass shifts suitable for particular analytical applications.

As illustrated in FIG. 1, the present disclosure provides for the strategic introduction of mass-tagged nucleosides at specific structural regions of synthetic mRNA molecules 100, including a mass-tagged capping agent comprising isotopically labeled guanosine 105A in the 5′ guanosine cap 105, a percentage of mass-tagged nucleosides such as pseudouridine 110A in the open reading frame 110, and a percentage of mass-tagged adenosine 115A in the poly-A tail 115. In some instances, the relative ratio between these non-naturally occurring mass-tagged nucleosides can inform on the manufacturing product's integrity and purity, representing critical quality attributes that may be measured consistently throughout the lifecycle of the RNA product. The present disclosure contemplates that libraries of stable isotopically labeled and modified nucleosides, including both canonical and non-canonical species, can enable quantitative analyses of oligonucleotide composition by mass spectrometry. In a preferred embodiment, solvent-assisted mechanochemical approaches may be configured to provide efficacious syntheses of isotopically labeled nucleosides, nucleotides, and dinucleotides, as well as short oligonucleotides suitable for use as reference materials. The astute introduction of mass-tagged nucleotides in RNA sequences may facilitate the detection and characterization of low abundance synthetic contaminants that could otherwise be difficult to distinguish from the intended product. Such approaches can provide improved means for benchmarking RNA products' quality assurance and quality control protocols for RNA-omics analyses and RNA sequence product quality and integrity monitoring.

In some preferred embodiments, the present disclosure provides methods that may generate tractable nucleic acids and provide access to certifiable reference materials that can help assure measurement quality across different analytical platforms and laboratories. As illustrated in FIG. 5, the synthesis and analysis workflow for mass-tagged RNA molecules may encompass the production of isotopically labeled nucleosides at various scales, from approximately 1 gram scale library production using ball milling apparatus to approximately 100-gram scale library production using planetary milling apparatus. The building blocks for RNA synthesis, including labeled and modified nucleoside phosphoramidites and labeled and modified nucleoside triphosphates, can be incorporated into RNA molecules that may subsequently be analyzed by complementary techniques such as Nanopore Direct RNA sequencing and LC-MS RNA-omics analysis. In another preferred embodiment, the dual analytical approach may enable comprehensive characterization of mass-tagged RNA molecules for quality assurance and quality control purposes, allowing verification of isotopic label incorporation and assessment of RNA product integrity. The present disclosure optionally contemplates that such innovative technologies can improve the protocols that support RNA products' quality assurance and quality control, which may be valuable for exploring and harnessing the complex field of epitranscriptomics and its therapeutic applications. Alternatively, the mass-tagged nucleosides may be employed in research applications to track nucleotide metabolism or to study RNA modification dynamics in biological systems.

### LC-MS Enabled RNA Composition Measurements Based on Label Incorporation

In a preferred embodiment, the present disclosure provides quantitative ribonucleoside LC-MS methods that may rely on chromatographic separation before detection by multiple reaction monitoring (MRM) mass spectrometry. These approaches can achieve limits of detection (LODs) down to approximately 60 attomole for some ribonucleosides when using standard mixtures with canonical and modified nucleosides at equal concentrations. Coelution of nucleosides may limit LC-MS/MS quantification because of signal suppression of less abundant nucleosides, which can make calibration curves non-linear and worsen quantifiability at concentrations relevant for mRNA analyses, such as when comparing m7-G versus A. As illustrated in FIG. 4, the use of isotopically labeled nucleosides that are isobaric to the RNA building blocks and separable by LC may be configured to address this analytical challenge. In some instances, if isotopic tags are introduced and differ from internal MS standards, then the absolute concentration and relative abundance of each nucleosidic component of the RNA construct can be accounted for with improved precision. The present disclosure thus enables innovative technologies that may improve protocols supporting RNA products' quality assurance (QA) and quality control (QC), which can facilitate exploration and harnessing of the complex field of epitranscriptomics and its therapeutic applications.

In some preferred embodiments, the utility of this method may be demonstrated by an exemplary embodiment involving mRNAs synthesized from ¹⁶O- and ¹⁸O-nucleoside triphosphates by a manufacturer incorporating specific isotopic payloads (% 160 vs. 180), which may be evaluated for their global isotopic/non-isotopic nucleosidic content by LC-MS in an MS facility. The RNA samples can then be isolated by precipitation, thoroughly rinsed with cold methanol, and analyzed according to established protocols. Approximately 1 nM of each synthesized RNA (UV quantified) may be digested to nucleotide monophosphates by nuclease P1 and then dephosphorylated to ribonucleosides by bacterial alkaline phosphatase. The nucleosidic mixture can be separated, and isobaric nucleosides may be quantified using MRM (exact mass and ¹⁸O-exact mass) on a LC-nano (C18 column) combined with a Q-Exactive orbitrap system. In some instances, a minimum % isotopic payload may be determined that can validate greater than 99% nucleoside composition using mass-tagged ¹⁸O-m7-GpppN, ¹⁸O-ATP, and ¹⁸O-N1-methyl-pseudo-UTP. This approach can generate tractable nucleic acids and provide access to certifiable reference materials that may help assure measurement quality throughout the product lifecycle.

In another preferred embodiment, the relative abundance of ¹⁸O-m7-G, ¹⁸O-G (which may represent degradation of ¹⁸O-m7-G), ¹⁸O-A, and ¹⁸O-N1-methyl-pseudo-U can be proportionate to the initial isotopic payload of the three tagged nucleotides. The relative abundance of (¹⁸O-m7-G+¹⁸O-G) to ¹⁸O-A may inform on the average poly-A tail 115 length, while the relative abundance of (¹⁸O-m7-G+¹⁸O-G) to ¹⁸O-N1-methyl-pseudo-U may inform on the efficiency of RNAP at producing the full ORF transcript since N1-methyl-pseudo-U can be found in the open reading frame 110. As illustrated in FIG. 1, the mRNA molecule 100 may comprise a 5′ guanosine cap 105 containing isotopically labeled guanosine 105A, an open reading frame 110 containing pseudouridine 110A, and a poly-A tail 115 containing isotopically labeled adenosine 115A. The relative ratio between these non-naturally occurring mass tagged nucleosides can inform on the manufacturing product's integrity and purity, representing critical quality attributes (CQAs) that may be measured in the same manner throughout the life cycle of the RNA product. Furthermore, the relative abundance of ¹⁸O-oxo-G vs. ¹⁸O-m7-G may inform on the oxidation of m7-G during sample processing, providing additional quality metrics. Alternatively, other isotopic labels such as ¹³C or ¹⁵N may be employed to achieve similar analytical objectives.

The present disclosure provides that astute introduction of mass-tagged nucleotides in RNA sequences, such as mRNA vaccines or miRNA therapeutics, may facilitate the detection and characterization of low abundance synthetic contaminants. In some preferred embodiments, this approach can provide improved means for benchmarking RNA products' QA/QC protocols for RNA-omics analyses and RNA sequence product quality and integrity monitoring. Libraries of stable isotopically labeled and/or modified nucleosides may be configured to enable quantitative analyses of oligonucleotides' composition by mass spectrometry with enhanced sensitivity and specificity. The solvent-assisted mechanochemical approaches developed for efficacious syntheses of nucleosides, nucleotides, and dinucleotides as well as short oligonucleotides can be applied to the production of isobaric isotopically labeled, canonical and non-canonical as well as modified nucleosides and nucleotides. In some instances, this methodology may optionally be extended to facilitate RNA product authentication and integrity verification across various therapeutic applications. The approach can generate tractable nucleic acids and provide access to certifiable reference materials that may help assure measurement quality throughout the manufacturing and distribution process.

LC-MS² and MALDI-ToF² Enabled RNA Sequence Integrity Measurements Based on Mass-Tagged Oligomers Used as Internal Standards

In a preferred embodiment, the present disclosure provides methods for evaluating the sequence of an in vitro RNA transcript using mass spectrometry techniques such as MALDI-ToF/ToF or LC-MS/MS. The analysis may be configured to sequence fragments of approximately 10-50 base pairs at a time, which can facilitate detailed characterization of the RNA product. For larger RNA molecules, partial hydrolysis to smaller fragments may be employed, allowing this approach to be implemented in a manner analogous to techniques widely used in proteomics applications. In some instances, quantitative measurements of short nucleic acid oligomers by mass spectrometry can be substantially free of experimental biases when a known amount of an isotopic form of the species of interest is added prior to sample processing and analysis. As illustrated in FIG. 4, these approaches may be configured to report the relative abundance of oligomeric fragments from a given RNA sample and can reveal the percentage of full-length transcript present in the sample. The integration of mass-tagged nucleosides into the RNA product, including a mass-tagged capping agent based on guanosine, a percentage of mass-tagged nucleosides within the functional sequence, and a percentage of mass-tagged adenosine in the poly-A tail 115, can provide a means for monitoring product integrity throughout the life cycle of the RNA therapeutic.

In another preferred embodiment, a test mRNA molecule 100 comprising isotopically labeled nucleosides as described herein may be treated with a low concentration of nuclease P1 for varying time periods, such as 15, 30, 60, or 120 minutes, before direct MALDI-ToF/TOF mass spectrometric measurements are performed. The conditions can be optimized to detect manageable fragment sizes, for instance fragments of approximately 6-10 nucleotides in length, using negative ionization mode with mass-to-charge ratios between approximately 1000 and 5000. Once suitable fragments are detected, these fragments may be targeted and their sequences determined, with selected fragments optionally authenticated by LC-MS/MS analysis. In some preferred embodiments, fragments that bridge the untranslated region (UTR) to the open reading frame 110 and the open reading frame 110 to the poly-A tail 115, and that are unique within the mRNA transcript, can be selected as reporter sequences for quality control purposes. As illustrated in FIG. 1, the strategic placement of isotopically labeled nucleosides at the 5′ guanosine cap 105, within the open reading frame 110, and at the poly-A tail 115 can facilitate the detection and characterization of low abundance synthetic contaminants while providing improved means for benchmarking RNA product quality assurance and quality control protocols. The relative ratio between non-naturally occurring mass-tagged nucleosides may inform on the manufacturing product's integrity and purity, representing a measurable attribute that can be monitored consistently throughout the product life cycle.

In some instances, a ¹³C-U-ribose mass-tagged oligonucleotide version of selected reporter fragments may be synthesized according to the mechanochemical processes described herein, and these isotopologous nucleotides can then be used as internal standards for quantification by LC-MS following nuclease P1 treatment. Under optimized conditions, if the mRNA sample contains predominantly full-length transcripts, the relative abundance of the bridging fragments may approach a one-to-one ratio once normalized to the added isotopologous synthetic fragments used as internal standards. Alternatively, deviations from this expected ratio can indicate the presence of truncated transcripts, degradation products, or contaminants within the sample. In some preferred embodiments, the analytical conditions may be optimized through iterative refinement, examining variables such as P1 nuclease concentration, treatment duration, and fragment selection criteria. The present disclosure contemplates that libraries of stable isotopically labeled and modified nucleosides can enable quantitative analyses of oligonucleotide composition by mass spectrometry, thereby facilitating RNA product authentication and integrity verification. This approach may generate tractable nucleic acids and can provide access to certifiable reference materials that help assure measurement quality for RNA-omics analyses and RNA sequence product monitoring applications, including mRNA vaccines and miRNA therapeutics.

### Limit of Detection of Tracers in RNA Sequence for Supply Chain Validation

The present disclosure addresses the growing need for innovative technologies that improve protocols supporting RNA products' quality assurance (QA) and quality control (QC), particularly as the complex field of epitranscriptomics and its therapeutic applications continues to expand. Each commercial RNA sequence or therapeutic RNA sequence may be different according to its application and purpose, such as mRNA vaccines encoding viral spike proteins, miRNA therapeutics targeting oncogenic pathways, or guide RNAs for CRISPR-based gene editing applications. In a preferred embodiment, once a particular sequence has been selected for manufacturing, consistency in its production process, purity, and stability can be monitored through the mass-tagging approaches described herein. The introduction of a mass-tagged capping agent based on guanosine, combined with a defined percentage of mass-tagged nucleosides present in the RNA functional sequence and a percentage of mass-tagged adenosine in the poly-A tail 115, may provide a comprehensive framework for assessing product integrity. In some instances, the relative ratio between non-naturally occurring mass-tagged nucleosides can inform on the manufacturing product's integrity and purity, representing critical quality attributes (CQAs) that may be measured consistently throughout the life cycle of the RNA product. As illustrated in FIG. 1, the mRNA molecule 100 can incorporate isotopically labeled guanosine 105A in the 5′ guanosine cap 105, pseudouridine 110A in the open reading frame 110, and isotopically labeled adenosine 115A in the poly-A tail 115, thereby enabling region-specific quality assessment.

In a preferred embodiment, computational analyses of experimental outcomes may reveal parameters that make one approach, such as RNA composition measurement versus RNA sequence integrity measurement, more cost and time-effective than the other for a given nucleic acid construct. For instance, a short therapeutic oligonucleotide of approximately 25 nucleotides may benefit from direct sequence integrity measurement, while a longer mRNA construct of several thousand nucleotides may be more efficiently assessed through composition analysis of its constituent regions. In another preferred embodiment, the reporting capabilities of mRNA products 100 incorporating isotopically labeled tracers through nucleosides' global analyses can be compared against non-tagged RNA monitored with selected isotopologous RNA oligomers. The present disclosure contemplates that libraries of stable isotopically labeled and modified nucleosides may enable quantitative analyses of oligonucleotides' composition by mass spectrometry, thereby facilitating RNA product authentication and integrity verification. In some preferred embodiments, these methods may be configured to be compared with state-of-the-art quality control processes based on standard LC-MS to establish relative performance metrics. Both evaluations can utilize the disclosed mechanochemical chemistries and the disclosed MS approaches, optionally incorporating additional analytical techniques as appropriate for specific applications.

As illustrated in FIG. 5, a preferred embodiment depicts procedures for synthesizing isotopically labeled nucleotides using solvent-assisted mechanochemical approaches, using them to generate mRNAs 100, and utilizing said nucleotides and mRNAs for the purposes of standards and quality control for RNA-omics with LC-MS. The astute introduction of mass-tagged nucleotides in RNA sequences, such as mRNA vaccines or miRNA therapeutics, may facilitate the detection and characterization of low abundance synthetic contaminants that might otherwise escape detection by conventional analytical methods. In some instances, this approach can provide improved means for the benchmarking of RNA products' QA/QC protocols for RNA-omics analyses and RNA sequence product quality and integrity monitoring. The present disclosure contemplates that the disclosed methodologies may generate tractable nucleic acids and provide access to certifiable reference materials that help assure measurement quality across different manufacturing batches and facilities. In another preferred embodiment, the mRNAs 100 generated using the aforementioned methodologies can be sequenced using direct RNA sequencing technologies. For instance, Nanopore technologies may be configured to cross-check the sequences assessed by LC-MS, providing orthogonal validation of product identity and integrity.

In some preferred embodiments, the cross-checking between mass spectrometry and direct RNA sequencing may be conducted during the initial optimization of the RNA synthesis and quality assurance methodologies to establish baseline performance parameters. Alternatively, this cross-checking can be conducted periodically on a subset of quality assurance analyses to ensure QA/QC remains robust and effective over extended production campaigns. The present disclosure recognizes that different therapeutic applications may warrant different frequencies of cross-validation, with high-risk applications such as personalized cancer vaccines potentially benefiting from more frequent verification. In a preferred embodiment, the integration of mass-tagged nucleotides with complementary sequencing technologies may be configured to provide a comprehensive quality management system for RNA therapeutics manufacturing. The solvent-assisted mechanochemical approaches developed for the efficacious syntheses of nucleosides, nucleotides, and dinucleotides as well as short oligonucleotides can be applied to produce isobaric isotopically labeled canonical, non-canonical, and modified nucleosides and nucleotides at scales suitable for industrial quality control applications. In some instances, the disclosed approaches may optionally be adapted to accommodate emerging RNA therapeutic modalities, such as circular RNAs or self-amplifying RNAs, by incorporating appropriate mass-tagged building blocks at structurally informative positions within these molecules.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For instance, the implementations described above can be directed to various combinations and subcombinations of the disclosed features and/or combinations and subcombinations of several further features disclosed above. In addition, the logic flow depicted in the accompanying figures and/or described herein do not necessarily require the particular order shown, or sequential order, to achieve desirable results. It will be readily understood to those skilled in the art that various other changes in the details, materials, and arrangements of the parts and method stages which have been described and illustrated in order to explain the nature of this inventive subject matter can be made without departing from the principles and scope of the inventive subject matter.

What we claim is:

1. A synthetic mRNA comprising one or more of:
a 5′ guanosine cap comprising a first isotopically labeled nucleoside;
an open reading frame comprising a second isotopically labeled nucleoside; and
a 3′ poly-A tail comprising a third isotopically labeled nucleoside,
wherein a ratio of said first, second, and third isotopically labeled nucleosides to their respective unlabeled nucleosides is between 1:10 and 1:1000.

2. The synthetic mRNA of claim 1, wherein said first isotopically labeled nucleoside comprises an ¹⁸O-labeled guanosine.

3. The synthetic mRNA of claim 2, wherein said ¹⁸O-labeled guanosine is an ¹⁸O-m7-guanosine.

4. The synthetic mRNA of claim 1, wherein said second isotopically labeled nucleoside comprises an ¹⁸O-labeled pseudouridine.

5. The synthetic mRNA of claim 4, wherein said ¹⁸O-labeled pseudouridine is an 18O-N1-methyl-pseudouridine.

6. The synthetic mRNA of claim 1, wherein said third isotopically labeled nucleoside comprises an 18O-labeled adenosine.

7. The synthetic mRNA of claim 1, wherein said first, second, and third isotopically labeled nucleosides each comprise an isotope selected from the group consisting of 18O, 13C, and 15N.

8. The synthetic mRNA of claim 1, wherein said 5′ guanosine cap comprises a structure selected from the group consisting of m7-GpppG, m7-GpppN, and m7-GpppN with a 2′-O-methyl modification.

9. The synthetic mRNA of claim 1, wherein said open reading frame encodes a polypeptide selected from the group consisting of a viral antigen, a therapeutic protein, and a reporter protein.

10. A method of synthesizing an isotopically labeled mRNA molecule, comprising:
synthesizing a first isotopically labeled nucleoside by mechanochemical means;
converting said first isotopically labeled nucleoside to a first isotopically labeled nucleoside triphosphate;
synthesizing a second isotopically labeled nucleoside by mechanochemical means;
converting said second isotopically labeled nucleoside to a second isotopically labeled nucleoside triphosphate;
synthesizing a third isotopically labeled nucleoside by mechanochemical means;
converting said third isotopically labeled nucleoside to a third isotopically labeled nucleoside triphosphate; and
incorporating said first, second, and third isotopically labeled nucleoside triphosphates into a synthetic mRNA molecule comprising a 5′ guanosine cap, an open reading frame, and a 3′ poly-A tail.

11. The method of claim 10, wherein said mechanochemical means comprises ball milling.

12. The method of claim 11, wherein said converting said first isotopically labeled nucleoside to a first isotopically labeled nucleoside triphosphate comprises:
reacting said first isotopically labeled nucleoside with POCl3 under ball milling conditions to form a dichlorophosphate intermediate; and
converting said dichlorophosphate intermediate to said first isotopically labeled nucleoside triphosphate via a methyl-imidazolate pathway.

13. The method of claim 10, wherein said first isotopically labeled nucleoside comprises an 18O-labeled guanosine, said second isotopically labeled nucleoside comprises an 18O-labeled pseudouridine, and said third isotopically labeled nucleoside comprises an ¹⁸O-labeled adenosine.

14. The method of claim 10, further comprising purifying said first, second, and third isotopically labeled nucleoside triphosphates by C18-reversed phase chromatography prior to said incorporating.

15. The method of claim 10, wherein said incorporating comprises using RNA polymerase to transcribe a DNA template in the presence of said first, second, and third isotopically labeled nucleoside triphosphates.

16. A method of conducting quality control for synthetic mRNA, comprising:
providing a synthetic mRNA molecule, wherein said synthetic mRNA molecule comprises isotopically labeled nucleosides in a 5′ guanosine cap, an open reading frame, and a 3′ poly-A tail;
fragmenting said synthetic mRNA molecule;
analyzing a fragmented synthetic mRNA molecule by mass spectrometry to acquire mass spectrometry data;
determining from said mass spectrometry data a relative abundance of said isotopically labeled nucleosides in said 5′ guanosine cap, said open reading frame, and said 3′ poly-A tail; and
comparing said relative abundance of said isotopically labeled nucleosides to an internal standard, wherein said internal standard comprises data regarding a relative abundance of said isotopically labeled nucleosides compared with their unlabeled counterparts during synthesis of said synthetic mRNA molecule.

17. The method of claim 16, wherein said fragmenting comprises treating said synthetic mRNA molecule with nuclease P1 to generate oligonucleotide fragments.

18. The method of claim 17, wherein said analyzing comprises liquid chromatography-mass spectrometry (LC-MS) or matrix-assisted laser desorption/ionization time-of-flight mass spectrometry (MALDI-TOF MS).

19. The method of claim 18, wherein said determining from said mass spectrometry data comprises quantifying said isotopically labeled nucleosides using multiple reaction monitoring (MRM).

20. The method of claim 16, further comprising detecting a presence of contaminants or degradation products in said synthetic mRNA molecule based on a deviation of said relative abundance of said isotopically labeled nucleosides from said internal standard.
